(19) United States
(12) Patent Application Publication
(10) Pub. No.: US 2009/0299269 A1
(43) Pub. Date: Dec. 3, 2009

(54) VASCULAR STIMULATION TO AID INTRAVASCULAR CELL REPLACEMENT THERAPY

(76) Inventors: John Foley, Lino Lakes, MN (US); Jihong Qu, Maple Grove, MN (US); Craig Stolen, New Brighton, MN (US); Roger Hastings, Maple Grove, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/473,033

(22) Filed: May 27, 2009

Related U.S. Application Data
(60) Provisional application No. 61/057,062, filed on May 29, 2008.

(51) Int. Cl. A61M 37/00

(52) U.S. Cl. 604/21

(57) ABSTRACT

A system and method are described involving the use of a cell delivery catheter that incorporates a stimulating electrode to promote vasodilation prior to injection of cells at a target location within a blood vessel. The vasodilation may be produced locally and/or distally from the target location near the target organ. The technique may be applied not only to intra-coronary injection of cells to treat heart disease but to injection of cells in any blood vessel that feeds a target organ.

### CROSS-REFERENCE TO RELATED APPLICATION

This application claims the benefit of U.S. Provisional Application No. 61/057,062, filed on May 29, 2008, under 35 U.S.C. §119(e), which is hereby incorporated by reference.

### FIELD OF THE INVENTION

This invention pertains to apparatus and methods for the treatment of heart disease and other diseases with therapeutic cells.

### BACKGROUND

Intracoronary injection of therapeutic cells has shown promise for the treatment of cardiac diseases. However, injection of large cell types (e.g. mesenchymal stem cells, myoblasts, etc.) can potentially clog capillary beds and lead to microembolisms. Reducing this risk requires delivering lower doses of cells and/or high injection pressures. Decreasing cell number can be expected to have a negative impact on treatment outcomes, and repeated injections have been shown to promote vessel spasms. Stenting can reduce the occurrence of spasms, but introduces other complications (e.g., restenosis) and increases cost. Thus, the number of cells that can be delivered using conventional approaches is hampered by having to balance low cell numbers with a small number of injections.

### BRIEF DESCRIPTION OF THE DRAWINGS

FIG. 1 illustrates a particular catheter with vascular stimulation capability.

FIG. 2 illustrates another particular catheter.

FIG. 3 depicts a system for delivering therapeutic cells in conjunction with vascular stimulation.

### DETAILED DESCRIPTION

Electrical stimulation within blood vessels can be used to promote their temporary dilation. Described herein is a system and method involving the use of cell delivery catheter that incorporates a stimulating electrode to promote vasodilation prior to injection of cells at a target location within a blood vessel. The vasodilation may be produced locally and/or distally from the target location near the target organ (e.g., at the arterioles feeding the target organ). The technique may be applied not only to intra-coronary injection of cells to treat heart disease but to injection of cells in any blood vessel that feeds a target organ. Examples of target organs besides the heart would include brain, liver, kidney, pancreas, or lung. The injected therapeutic cells may be any type that beneficially affects the target organ such as embryonic stem cells, induced pluripotent stem cells, somatic cell nuclear transfer derived stem cells, fetal stem cells, umbilical stem cells, adult stem cells, mesenchymal stem cells, hematopoietic stem cells, endothelial stem cells, peripheral blood stem cells, adipose derived stem cells, myoblasts, or cardiac progenitor cells The described technique decreases the problems associated with microembolisms, allows for the injection of larger numbers of cells, and reduces the need for multiple procedures. Other applications for this type of electrical stimulation may include promoting local dilation of blood vessels to help maneuver a catheter through complex arterial and venous systems, dilation of veins prior to pacing lead placement during implantation of a cardiac rhythm management device and using subsequent contraction to help anchor lead in place, and modifying the electrical stimulation to promote vasoconstriction that decreases blood flow during therapeutic cell and/or gene therapy vector injection.

A catheter for delivering vascular stimulation in conjunction with injection of therapeutic cells or other agents includes one or more stimulation electrodes at the distal portion of the catheter, a lumen through which the cells and/or agents may be injected, and one or more conductors incorporated into the catheter for connecting to a pulse generator in order to excite the stimulation electrode(s). The catheter may be a conventional over-the-wire type in which the catheter is advanced through the vasculature over a guide wire contained within a lumen of the catheter. The stimulation may be delivered as unipolar stimulation referenced to another electrode disposable at a surface or internal body location. For example, unipolar stimulation may be referenced to a skin patch electrode affixed to a surface location overlying the catheter stimulation electrode. Multiple electrodes may be used to deliver bipolar or multi-polar stimulation or to selectively deliver stimulation to multiple locations. The stimulation electrode(s) may be flush with, recessed from, or extend from the catheter surface. In another embodiment, the stimulation can be performed using a stent electrode, where the stent may have one or more leads and be retractable. The catheter may also incorporate a measurement electrode connectable to sensing circuitry for monitoring the voltage drop between the stimulating electrode and the blood.

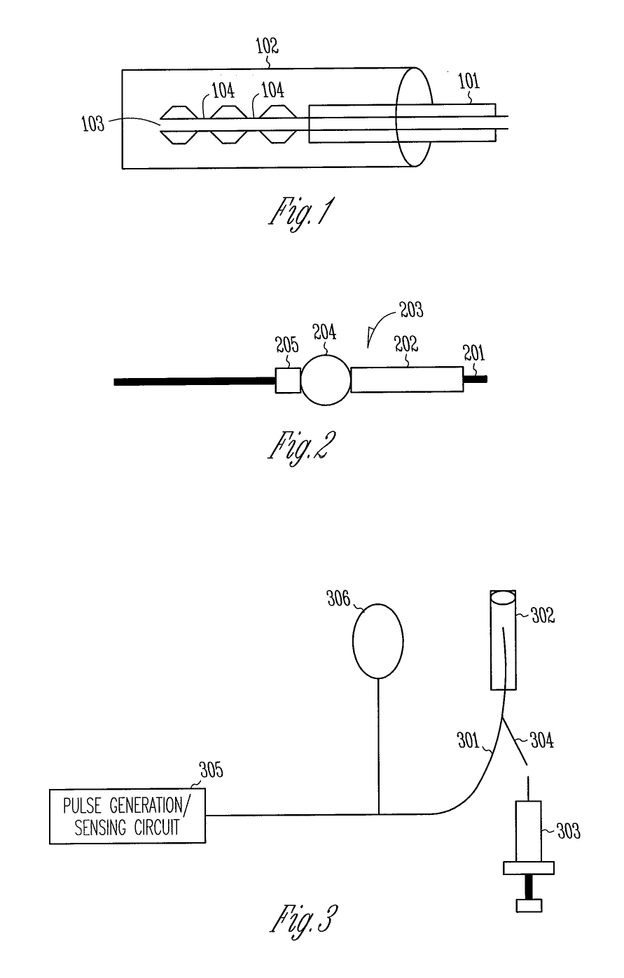

FIG. 1 illustrates one particular embodiment of a catheter that incorporates a stimulation electrode. A catheter 101 is shown as being disposed with a blood vessel 102. The catheter has a lumen 103 allowing therapeutic cells and/or other agents to be injected into the blood stream out of the distal end of the catheter. The catheter in this embodiment has multiple stimulation electrodes 104 recessed from the catheter surface.

FIG. 2 shows another particular embodiment of a catheter 201 that incorporates a tubular platinum stimulation electrode 202 placed near the distal end of the catheter and sized to slide over the catheter tubing. An insulated lead 203 from the electrode 202 exits the proximal hub of the catheter. An epoxy ball 204 at the proximal end of the electrode provides electrical insulation of the solder connection to the lead wire incorporated into the catheter. A small coil 205 proximal to the epoxy ball is a measurement electrode used to monitor the voltage drop between the stimulation electrode and the blood.

FIG. 3 depicts a system for delivering therapeutic cells and/or other agents with vascular stimulation. The catheter 301 is shown as being disposed within a blood vessel 302 at a target location. A syringe 303 is used to inject therapeutic cells and/or other agents into a catheter port 304 continuous with a lumen within the catheter 301 for delivering the cells out of the distal end. Pulse generation/sensing circuitry 305 is connected to one or more stimulation and/or measurement electrodes of the catheter via one or more conductors within the catheter. A return patch electrode 306 is also provided for referencing the stimulation voltage and which may be placed on the skin of the patient near the target location. The pulse generation circuitry may be configurable to deliver electrical stimulation at a variable frequency range which, as described below, may have either vasodilative or vasoconstrictive effects.

An exemplary method for injection of therapeutic cells using a catheter as described above is as follows. The catheter is disposed in a blood vessel at a target location by, for example, fluoroscopically guiding the catheter using the over-the-wire technique. Electrical stimulation is then delivered to the blood vessel via a stimulation electrode at the distal portion of the catheter in order to cause vasodilation distal to the target location, and therapeutic cells are injected at the target location via the catheter. Electrical stimulation at the target location may be either anodic or cathodic, may be delivered as unipolar, bipolar, or multi-polar stimulation, and may be delivered as DC or as an AC waveform of any type (e.g., square-wave or sinusoidal). It has been found that DC or lower frequency waveforms are usually most appropriate for producing vasodilation. A possible mechanism for this effect is block of the adjacent sympathetic nerve. Once blocked, lack of adrenegric stimulation of the smooth muscle of blood vessels distal to the site of block results in dilation of vessels fed by the artery and adjacent nerve. The blood vessel at which the stimulation is applied may also be dilated due to the membrane potential of the smooth muscle becoming hyperpolarized and/or from lack of nerve stimuli. Vasoconstriction (or spasm) after injection of the cells may by produced by increasing the frequency content of the stimulating waveform to something in excess of about 3 Hz (either polarity). The vasoconstriction occurs at the site of the stimulation electrode in direct response to the stimulation. It may also be preferable to sense the cardiac cycle of the patient and for the stimulation energy to be applied so as to avoid stimulation during the relative refractory period of the T-wave phase when the myocardium is vulnerable to fibrillation. For vasodilation, this could be direct current applied during systole, but with rounded rise and fall times (e.g., a sinusoidal waveform) to avoid high frequency content. Vasoconstriction current can be applied at frequencies above 3 Hz during systole. After injection of the therapeutic cells, the frequency of the electrical stimulation may be increased as described above to cause vasoconstriction at the target location. The stimulating electrode may also be used to cause vasodilation while maneuvering the catheter to the target location.

The invention has been described in conjunction with the foregoing specific embodiments. It should be appreciated that those embodiments may also be combined in any manner considered to be advantageous. Also, many alternatives, variations, and modifications will be apparent to those of ordinary skill in the art. Other such alternatives, variations, and modifications are intended to fall within the scope of the following appended claims.

What is claimed is:

1. A method, comprising:
disposing a catheter in a blood vessel at a target location;
delivering electrical stimulation to the blood vessel via a stimulation electrode at the distal portion of the catheter in order to cause vasodilation distal to the target location; and,
injecting therapeutic cells at the target location via the catheter.

2. The method of claim 1 further comprising increasing the frequency of the electrical stimulation to cause vasoconstriction at the target location subsequent to injection of the cells.

3. The method of claim 1 further employing the stimulating electrode to cause vasodilation while maneuvering the catheter to the target location.

4. The method of claim 1 wherein the target location is within a coronary artery.

5. The method of claim 1 wherein the catheter incorporates a tubular platinum electrode placed near the distal end of the catheter and sized to slide over the catheter tubing.

6. The method of claim 1 further comprising placing a return patch electrode on the skin of the patient near the target location.

7. The method of claim 1 wherein the catheter incorporates multiple electrodes excitable by separate conductors within the catheter.

8. The method of claim 2 wherein the multiple electrodes are recessed from the surface of the catheter.

9. The method of claim 1 further comprising employing a measurement electrode incorporated into the catheter to monitor the voltage drop between the stimulating electrode and the blood.

10. The method of claim 1 wherein the target location is such that the therapeutic cells are delivered to the heart, brain, liver, kidney, pancreas, or lung.

11. The method of claim 1 wherein the therapeutic cells are selected from a group that includes embryonic stem cells, fetal stem cells, umbilical stem cells, adult stem cells, mesenchymal stem cells, hematopoietic stem cells, endothelial stem cells, peripheral blood stem cells, adipose derived stem cells, myoblasts, and cardiac progenitor cells.

12. The method of claim 1 further comprising sensing the cardiac cycle of the patient and timing electrical stimulation delivery to avoid the vulnerable T-wave phase.

13. An apparatus for delivery of therapeutic cells and/or other therapeutic agents, comprising:
a catheter adapted for disposition at a target location within a blood vessel and having a lumen through which the cells and/or agents may be injected;
one or more stimulation electrodes at the distal portion of the catheter for delivering electrical stimulation at the target location; and,
one or more conductors incorporated into the catheter for connecting to a pulse generator in order to excite the stimulation electrode(s).

14. The apparatus of claim 13 further comprising pulse generation circuitry configurable to deliver the electrical stimulation at a variable frequency range.

15. The apparatus of claim 13 further comprising a skin patch electrode affixable to a surface location overlying the catheter stimulation electrode for providing a return path for current from the stimulation electrode.

16. The apparatus of claim 13 wherein the stimulation electrode(s) are flush with the catheter surface.

17. The apparatus of claim 13 wherein the stimulation electrode(s) are recessed from the catheter surface.

18. The apparatus of claim 13 wherein the stimulation electrode(s) extend from the catheter surface.

19. The apparatus of claim 13 wherein the stimulation electrode(s) is a stent electrode.

20. The apparatus of claim 13 further comprising a measurement electrode connectable to sensing circuitry for monitoring the voltage drop between the stimulating electrode and the blood.
